SEQUENCE LISTING (6 sequences)

SEQ ID NO: 1 - protein, 8 aa, Homo sapiens
CVFPKESD

SEQ ID NO: 2 - protein, 8 aa, Artificial sequence
  Synthetic peptide
CAFPKESD

SEQ ID NO: 3 - protein, 8 aa, Artificial sequence
  Synthetic peptide
CVFPRESD

SEQ ID NO: 4 - protein, 8 aa, Artificial sequence
  Synthetic peptide
CVFPKDSD

SEQ ID NO: 5 - protein, 8 aa, Artificial sequence
  Synthetic peptide
CVFPKETD

SEQ ID NO: 6 - protein, 8 aa, Artificial sequence
  Synthetic peptide
CVYPKESD

(19) United States
(12) Patent Application Publication
(10) Pub. No.: US 2022/0252583 A1
(43) Pub. Date: Aug. 11, 2022

(54) SEMICONDUCTOR BASED BIOSENSOR UTILIZING THE FIELD EFFECT OF A NOVEL COMPLEX COMPRISING A CHARGED NANOPARTICLE

(71) Applicant: LIFE SCIENCE INKUBATOR SACHSEN GMBH & CO. KG, Dresden (DE)

(72) Inventors: Tom STUECKEMANN, Dresden (DE); Norman GERSTNER, Dresden (DE)

(21) Appl. No.: 17/615,285

(22) PCT Filed: Jun. 2, 2020
(86) PCT No.: PCT/EP2020/065163

§ 371 Date: Nov. 30, 2021

(30) Foreign Application Priority Data
May 31, 2019 (EP) 19177628.5

(51) Int. Cl. G01N 33/543; G01N 33/542; G01N 33/58; G01N 27/414

(52) U.S. Cl. CPC G01N 33/5438; G01N 33/542; G01N 2333/4737; G01N 27/4145; G01N 27/4146; G01N 33/587

(57) ABSTRACT

The present invention relates to a biosensor for detecting analytes comprising a bio-sensing surface which comprises a field effect transistor and a first binding molecule which is bonded to the surface of the field effect transistor. Furthermore, the biosensor comprises a complex comprising second binding molecules which are conjugated to charged nanoparticles by linker molecules, wherein at least one second binding molecule conjugated to a charged nanoparticle interacts with the first binding molecule wherein the charged nanoparticle is configured to apply a field effect on the field effect transistor. Moreover, the present invention provides a method of detecting an analyte by a biosensor.

The present invention relates to a biosensor for detecting analytes comprising a bio-sensing surface which comprises a field effect transistor and a first binding molecule which is bonded to the surface of the field effect transistor. Furthermore, the biosensor comprises a complex comprising second binding molecules which are conjugated to charged nanoparticles by linker molecules, wherein at least one second binding molecule conjugated to a charged nanoparticle interacts with the first binding molecule wherein the charged nanoparticle is configured to apply a field effect on the field effect transistor. Moreover, the present invention provides a method of detecting an analyte by a biosensor.

### BACKGROUND OF THE INVENTION

Biosensors include a biological receptor linked on an electrical transducer in such a way that biological interactions are translated into electrical signals^{1,2}. Semiconductor based Field Effect Transistors (FETs) have received significant attention as highly sensitive transducers suitable for building fast and inexpensive diagnostic devices³⁻¹². However the ability of FETs to measure all relevant analytes (biomarkers) with a great sensitivity in physiological solutions like blood, serum or plasma remains challenging due to the phenomenon of charge screening or Debye screening in high salt concentrations^{3,6,7,13}.

Several attempts to increase the sensitivity of FETs in physiological solutions have been made^{3,5-7,14,15}. The explored strategies can be categorized in four groups:

1. Material

Different semiconductor materials e.g. Carbon Nanotubes (CNTs)⁶, Graphen³, Si-Nanowires¹⁴ and many more have been used.

2. Modifications

Several sensor surface modifications have been described e.g. poly ethylene glycol (PEG) has been used in high ionic strength solutions to increase the sensitivity of CNTs and graphene^{3,6} or the US Patent Application (US2006/0205013) has used Pyrene groups on the sensor surface to induce charges of nuclear acids¹⁵.

3. Passivation

Several sensor surface passivation steps, to reduce current leakage from the source electrode to the drain electrode, through the applied sample, bypassing the semiconductor material, have been published. For CNT-FETs the US Patent (US 2012/0073992) has described polymers like Teflon, polydimethylsiloxane (PDMS), polymethylmethacrylate (PMMA), silicon dioxide (SiO2), or silicon nitride (SiN), whereas Filipiak et. al applied SU-8 2005, to reduce leakage current^{6,8}. For Si based transistors anti-adhesion protective molecules like poly ethylene glycol (PEG) terminated self-assembled monolayers, or benzene terminated self-assembled monolayers, have been described¹⁴.

4. Capture Molecule

Since the electric field strength falls as the inverse square of the distance between target and nanomaterial surface, the size of the capture molecule is important. Therefore different capture molecules like antibodies, antibody fragments, enzymes, nanobodies, aptamers or nucleic acids have been coupled on a diverse set of different semiconductor materials^{2,3,6,13,16,17}.

Despite of all those FET Biosensors optimization, the number of measurable analytes is very small. This means that some biomarkers can be detected very well by FET Biosensors whereas others can't. The main reasons behind this phenomena are the heterogenic physical/chemical properties among the individual biomarkers. Especially important for generating a field effect on the semiconductor material is the relative charge density of an analyte. The relative charge density is calculated by dividing [the number of surface charges at neutral pH] through [the diameter of the biomarker in nm]. Thereby small, but highly charged analytes like 21 mer miRNAs (21 charges/1 nm=21) generate a strong field effect and can be much easier detected compared to large, uncharged molecules like Interleukin 6 (5 charges/3 nm=1.67. Consequently, the size and the charge of an analyte are critical in order to generate a field effect. However, since biomarkers are selected for their clinical indication instead of their relative charge density, it is extremely challenging to build a biosensor, which can measure a range of diverse analytes. Therefore, no FET biosensor has been reported, which is suitable to measure most of the relevant biomarkers under physiological conditions.

It is the task of the present invention to provide a biosensor for detecting analytes, which overcomes the current limitations of semiconductor based biosensors.

This task is solved by the present invention by providing a biosensor for detecting analytes, which comprises
a bio-sensing surface, wherein the bio-sensing surface comprises a field effect transistor and a first binding molecule which is bonded to the surface of the field effect transistor.
and a complex, which comprises second binding molecules which are conjugated to charged nanoparticles by linker molecules;
   wherein at least one second binding molecule is conjugated to one charged nanoparticle and wherein the at least one second binding molecule conjugated to a charged nanoparticle interacts with the first binding molecule wherein the charged nanoparticle is configured to apply a field effect on the field effect transistor. Furthermore, the affinity of the second binding molecule to the first binding molecule is adaptable such that the first binding molecule releases the complex comprising the second binding molecule in presence of the analyte and the current measured in dependence of a voltage applied to the field effect transistor is changed due to displacement of the complex comprising the second binding molecule by the analyte.

In the overall context of the invention, the wording ‘low affinity molecule’ or ‘second binding molecule’ means a molecule contained in the complex of the invention which is able to bind to the first binding molecule of the bio-sensing surface. Further characteristics of the low affinity molecules or second binding molecule are described below.

Furthermore, the present invention provides a method of detecting an analyte by a biosensor, said method comprising a biosensor, wherein said biosensor comprises a bio-sensing surface which comprises a field effect transistor and a first binding molecule which is bonded to the surface of the field effect transistor. Furthermore, the biosensor comprises a complex comprising second binding molecules which are conjugated to charged nanoparticles by linker molecules. According to the invention the method comprises the following steps
i. selecting a second binding molecule with a lower affinity to the first binding molecule compared to the analyte;
ii. conjugating the second binding molecules to charged nanoparticles;
iii. bonding the second binding molecules which are conjugated to charged nanoparticles to the bio-sensing surface;
iv. measuring the field effect of the charged nanoparticles to the field effect transistor by measuring the current in dependence of a voltage applied to the field effect transistor;
v. contacting the analyte with the bio sensing surface and the charged nanoparticles which are conjugated to second binding molecules;
vi. measuring the change of the field effect acting on the field effect transistor by measuring the current in dependence of a voltage applied to the field effect transistor, wherein the second binding molecules conjugated to charged nanoparticles are partially or completely displaced by analytes due to the higher affinity of the analytes to the first binding molecules, thereby changing the field effect acting on the field effect transistor.

In a preferred embodiment, the invention provides a method of detecting an analyte with a biosensor, wherein the method comprises the steps of
i. providing a biosensor with a bio-sensing surface which comprises a field effect transistor and a first binding molecule which is bonded to the surface of the field effect transistor;
ii. selecting a second binding molecule with a lower affinity to the first binding molecule compared to the analyte;
iii. conjugating the second binding molecules to charged nanoparticles via linker molecules;
iv. bonding the second binding molecules, which are conjugated to charged nanoparticles via linker molecules, to the first binding molecule of the bio-sensing surface;
v. measuring the field effect of the charged nanoparticles to the field effect transistor by measuring the current in dependence of a voltage applied to the field effect transistor;
vi. contacting the analyte with the bio-sensing surface and the charged nanoparticles which are conjugated to second binding molecules;
vii. measuring the change of the field effect acting on the field effect transistor in presence of the analyte by measuring the current in dependence of a voltage applied to the field effect transistor,
   wherein the second binding molecules conjugated to charged nanoparticles are partially or completely displaced by analytes due to the higher affinity of the analytes to the first binding molecules, thereby changing the field effect acting on the field effect transistor.

According to the invention the semiconductor surface is modified with a binding molecule with which the second binding molecule interacts in such a way that the charged nanoparticles can apply a field effect on the semiconductor material. The affinity of the second binding molecules is selected in such a way that the first binding molecule can release the second binding molecule in the presence of an analyte but does not significantly release the second binding molecule in absents of an analyte. Because the analyte triggers the release of the second binding molecule the conjugated charge carrying nanoparticle cannot longer apply a field effect on the semiconductor material. Thereby the applied field effect on the semiconductor material can be directly correlated to the analyte concentration applied to the sensor and can be electrically read out by the current measured in dependence of a voltage applied to the field effect transistor.

The biosensor and method of the invention are therefore based on the replacement or displacement of complexes comprising conjugates of second binding or low affinity molecules and nanoparticles from a first binding molecule as described above by analytes with higher affinity to the first binding molecule and the resulting changes in the field effect by said replacement or displacement, which can be measured with high sensitivity. Several attempts of optimizing field effect transistor-based biosensors using conjugates of biomolecules and nanoparticles have been made″, but the displacement approach of the present invention has not been used so far.

### DETAILED DESCRIPTION OF THE INVENTION

The biosensor according to the invention is based on a bio-sensing surface and a complex. The bio-sensing surface comprises a field effect transistor and a first binding molecule which is bonded to the surface of the field effect transistor.

In one embodiment of the invention the first binding molecule is selected from proteins, peptides, nucleic acids, antibodies and fragments thereof including monoclonal antibodies, humanized forms of non-human antibodies, single-chain Fv or sFv antibody fragments, diabodies or isolated antibodies, preferably the first binding molecule is selected from antibodies and fragments thereof.

The term “antibody” is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity. The antibody may be an IgM, IgG (e.g. IgG1, IgG2, IgG3 or IgG4), IgD, IgA or IgE, for example.

“Antibody fragments” comprise a portion of an intact antibody, generally the antigen binding or variable region of the intact antibody. Examples of antibody fragments include Fab, Fab′, F(ab′)2, and Fv fragments: diabodies; single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to “polyclonal antibody” preparations which typically include different antibodies directed against different determinants (epitopes), each monoclonal antibody is directed against a single determinant on the antigen. In addition to their specificity, the monoclonal antibodies can frequently be advantageous in that they are synthesized by the hybridoma culture, uncontaminated by other immunoglobulins. The “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by generally well known recombinant DNA methods. The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain a minimal sequence derived from a non-human immunoglobulin. For the most part, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences.

These modifications are made to further refine and optimize antibody performance. In general, the humanized antibody will comprise substantially all or at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986), Reichmann et al, Nature. 332:323-329 (1988): and Presta, Curr. Op. Struct. Biel., 2:593-596 (1992). The humanized antibody includes a Primatized™ antibody wherein the antigen-binding region of the antibody is derived from an antibody produced by immunizing macaque monkeys with the antigen of interest.

“Single-chain Fv” or “sFv” antibody fragments comprise the VH and VL domains of an antibody, wherein these domains are present in a single polypeptide chain. Generally, the Fv polypeptide further comprises a polypeptide linker between the VH and VL domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VD) in the same polypeptide chain (VH-VD). By using a linker that is too short to allow pairing between the two domains on the same chain, the domains are forced to pair with the complementary domains of another chain and create two antigen-binding sites. Diabodies are described more fully in Hollinger et al., Proc. Natl. Acad. Sol. USA, 90:6444-6448 (1993). An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The bio-sensing surface further comprises a filed effect transistor (FET), wherein every field effect transistor known in the art is suitable to be used in the invention. Especially, CNT-FET (carbon nanotube based FET) like swCNT-FET (single walled carbon nanotube based FET) or mwCNT-FET (multi walled carbon nanotube based FET) furthermore silicon nanowire FETs or any other nanoscale semiconductor material are suitable. Preferably swCNT-FETs are used in the invention.

Further, the invention comprises a complex which comprises second binding molecules which are conjugated to charged nanoparticles by linker molecules.

According to the invention the complex comprises
a charged nanoparticle selected from a group comprising metallic nanoparticles, semiconductor nanoparticles, quantum dots or non-metallic nanoparticles, most preferable from metal nanoparticles, wherein the nanoparticles are charged to carry a positive or negative charge;
at least one linker molecule selected from a group comprising a bond, alkyl, polyethylene glycol (PEG), polyamide, peptide, carbohydrate, oligonucleotide or polynucleotide, most preferable from PEG; and
at least one second binding molecule selected from a group comprising proteins, peptides, nucleic acids or synthetic components, preferably from peptides.

In a preferred embodiment of the invention the charged nanoparticles are conjugated by a linker molecule to a second binding molecule, building the following structure:

A-L-B

wherein A is a second binding molecule, L is a linker molecule, and B is a charged nanoparticle.

In another embodiment of the invention more than one second binding molecule is conjugated to a charged nanoparticle. According to the invention 1 to 100, preferably 1 to 50 second binding molecules can be conjugated to one charged nanoparticle, wherein every second binding molecule is conjugated by a linker molecule.

In a preferred embodiment one second binding molecule is conjugated to one charged nanoparticle. In that event, consequently, one second binding molecule bound to one charged nanoparticle can be bound to one binding molecule on the bio-sensing surface following the laws of affinity as described below. If more than one second binding molecule is bound to one charged nanoparticle the laws of avidity take hold. Which means that multivalent bonds of on charged nanoparticle having several second binding molecules bound to the first binding molecules on the bio-sensing surface are possible. This could lead to higher association constants for the second binding molecule and the first binding molecule compared to the association constant if one second binding molecule bound to one charged nanoparticle is exclusively bound to one binding molecule. Nevertheless, surprisingly, according to the invention it has turned out that avidity effects can be neglected if the ratio of second binding molecule to charged nanoparticle is in the range of 1 charged nanoparticle to 1 to 100 second binding molecules.

The second binding molecule is selected from proteins, peptides, nucleic acids or synthetic components, preferably from peptides.

In biochemistry, affinity is a measure of the tendency of molecules to bind to other molecules. The association constant can be used to quantify the affinity between two binding partners, where the higher the affinity, the greater the association constant. Using the example of the formation of a complex ES from the binding partners E and S

$$E + {S}_{{←}_{k {1}^{′}}}^{{\to }^{k 1}} [ ES ] , \qquad ( 1 )$$

The association constant Kₐ is defined as

$$\begin{matrix}{K}_{a} = \frac{[ E S ]}{[ E ] * [ S ]} , & ( 2 ) \\ {K}_{a} = \frac{k 1}{k {1}^{′}} , respectively . & ( 3 )\end{matrix}$$

k₁ and k_{1′} are the rate constants for the association of E and S and the dissociation of the complex ES, respectively. Analog consideration can be carried out for the dissociation constant, which is the reciprocal of the association constant.

Since it turned out that avidity effects can be neglected for ratios of second binding molecules to charged nanoparticles as used according to the invention, even in case of multivalent bonds the association constant can be approximated according to the above definition. Meaning that for the purpose of the present invention the affinity constant for molecules with multivalent bonds can be approximated by the affinity constant for molecules with a single bond.

Basically, the association constant Kₐ of the second binding molecule and the first binding molecule is smaller compared to the association constant of the analyte and the first binding molecule. What is expressed in the following by the wording, the second binding molecule has a lower affinity than the analyte for binding to the first binding molecule. Furthermore, a lower affinity of the second binding molecule to the first binding molecule compared to the affinity of the analyte to the first binding molecule results in displacement of the second binding molecule from the binding site of the first binding molecule in presence of the analyte by the analyte.

Therefore, in a preferred embodiment of the invention the affinity of the second binding molecule to the first binding molecule is smaller compared to the affinity of the analyte to the first binding molecule.

It is crucial for the present invention that the affinity of the second binding molecule to the first binding molecule of the bio-sensing surface is less compared to the affinity of the analyte to the first binding molecule of the bio-sensing surface. This property guarantees that the complex is released in the presence of the analyte. Moreover, the affinity of the second binding molecule must be in a range that the second binding molecule is bound to the first binding molecule of the bio-sensing surface in absence of the analyte, so that the charged nanoparticle comprised in the complex can apply a field effect on the field effect transistor of the bio-sensing surface.

The second binding molecule may be further modified in such a way that the affinity for binding to the first binding molecule is altered, in particular reduced, compared to the intact analyte. Which means Kₐ of second binding molecule and first binding molecule is reduced compared to Kₐ of analyte and first binding molecule. Thereby the capability of the analyte to displace the complex captured at the binding site of the first binding molecule is improved. Altering, i.e. lowering of the affinity of the molecule captured at the antibody binding site may be achieved by point mutation, chemical modification by, e.g. biotinylation, glycosylation or any other method known in art.

In one embodiment of the invention the affinity of the second binding molecule is altered by point mutation, chemical modification by, e.g. biotinylation, glycosylation or any other method known in art.

In a preferred embodiment, the second binding molecule part of the complex captured at the binding site of the first binding molecule is a fragment of an antigen. The fragment of an antigen may be known in the art or is a synthetic peptide, wherein the amino acid sequence of a synthetic peptide is suitably adapted such that the binding to the binding site of the first binding molecule of the invention is facilitated.

In a more preferred embodiment, such antigen fragment or synthetic peptide has a chain length of 4 to 22 amino acids, more preferably of 5 to 15 amino acids, most preferably of 6 to 12 amino acids.

A further modification of the affinity of the second binding molecule to the first binding molecule of the bio-sensing surface by the above described measures has the advantage that the affinity of the second binding molecule to the first binding molecule of the bio-sensing surface can be regulated to be in an optimal range. Optimal range meaning that the second binding molecule is bound to the first binding molecule of the bio-sensing surface in absence of the analyte but is released in presence of the analyte.

The complex further comprises a linker molecule, which is selected from a bond, alkyl, polyethylene glycol (PEG), polyamide, peptide, carbohydrate, oligonucleotide or polynucleotide, most preferable from PEG.

The linker molecule is further characterized in that it has a Maleimide group on its proximal end and a NH₂ group or an NHS-Ester group or a Sulfo-NHS-Ester group on its distal end.

Therefore in a preferred embodiment of the invention the linker molecule has a Maleimide group on its proximal end and a NH₂ group or an NHS-Ester group or a Sulfo-NHS-Ester group on its distal end.

For example suitable linker molecules are selected from a group comprising Mal-PEG-NH2, Mal-PEG-SulfoNHS, Mal-PEG-NHS.

PEG is an oligomer or polymer composed of ethylene oxide monomers with the following monomer structure (—CH₂—CH₂—O—)ₙ. Because different applications require different polymer chain lengths, PEGs are prepared by polymerization of ethylene oxide and are commercially available over a wide range of molecular weights from 300 g/mol to 10,000,000 g/mol. While PEGS with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly (ethylene glycol), abbreviated mPEG. Lower-molecular-weight PEGs are also available as purer oligomers, referred to as monodisperse, uniform, or discrete.

PEGS are also available with different geometries:
Linear PEGs, where the ethylene oxide monomers are bound to each other in an unbranched polymer chain;
Branched PEGs, which have three to ten PEG chains emanating from a central core group;
Star PEGs, which have 10 to 100 PEG chains emanating from a central core group; and
Comb PEGs, which have multiple PEG chains normally grafted onto a polymer backbone.

The numbers that are often included in the names of PEGs indicate their average molecular weights (e.g. a PEG with n=9 would have an average molecular weight of approximately 400 daltons, and would be labeled PEG 400). Most PEGs include molecules with a distribution of molecular weights (i.e. they are polydisperse). The size distribution can be characterized statistically by its weight average molecular weight (Mw) and its number average molecular weight (Mn), the ratio of which is called the polydispersity index (Mw/Mn). Mw and Mn can be measured by mass spectrometry.

PEG is soluble in water, methanol, ethanol, acetonitrile, benzene, and dichloromethane, and is insoluble in diethyl ether and hexane.

In a preferred embodiment, the linker of the invention comprises a linear PEG. Using linear PEGS has the advantage that they are cheap and possess a narrower molecular weight distribution.

When linear PEG is used to form the linker of the conjugate of the invention, it has suitably a molecular weight in the range of 40 Da to 10,000 Da, preferably in the range of 200 Da to 6,000, more preferably in the range of 400 Da to 4,000 Da, most preferably in the range of 1,000 Da to 3,400 Da.

Furthermore, according to the invention charged nanoparticles are comprised in the complex, which are used as charge carrying objects, able to apply a proper field effect on the field effect transistor of the bio-sensing surface.

Charged nanoparticles are selected from a group comprising metallic nanoparticles, semiconductor nanoparticles, quantum dots or non-metallic nanoparticles, most preferable from metal nanoparticles, wherein the nanoparticles are charged to carry a positive or a negative charge. Suitable non-metallic nanoparticles are for example nanoparticles comprising carbides or nitrides, like aluminum nitride, boron nitride, boron carbide, silicon carbide, silicon nitride, titanium carbide, titanium nitride, tungsten carbide, tungsten nitride or zirconium carbide.

Furthermore, suitable non-metallic nanoparticles are for example oxides comprising Antimony(III) oxide, Antimon Tin Oxide (ATO), Aluminium Zinc Oxide (AZO), Barium titanate (BaTiO3), Bismuth(III) oxide (Bi2O3), Cerium(IV) oxide (CeO2), Chromium(III) oxide (Cr2O3), Cobalt(II, III) oxide (Co3O4), Copper(II) oxide (CuO), Dysprosium(III) oxide (Dy2O3), Erbium(III) oxide (Er2O3), Europium(III) oxide (Eu2O3), Gadolinium(III) oxide (Gd2O3), Hafnium(IV) oxide (HfO2), Indium(III) oxide (In2O3), Iron(II, III) oxide (Fe3O4), Indium Tin Oxide (ITO), Lanthanum(III) oxide (La2O3), Magnesium(II) oxide (MgO), Neodymium(III) oxide (Nd2O3), Nickel(II) oxide (NiO), Samarium(III) oxide (Sm2O3), Silicon(IV) oxide (SiO₂), Strontium titanate (SrTiO3), Tin(IV) oxide (SnO2), Titanium(IV) oxide (TiO2), Yttrium(III) oxide (Y2O3), Zinc oxide (ZnO), Zirconium(IV) oxide (ZrO2), α-Aluminium oxide (Al₂O₃), α-Iron(III) oxide (Fe2O3), γ-Aluminium oxide (Al₂O₃) or γ-Iron(III) oxide (Fe2O3).

Charged nanoparticles used in the complex of the invention have a molecular size in the range of 1-100 nm, preferably in the range of 5-50 nm, more preferably in the range of 5-40 nm, most preferably in the range of 5-20 nm.

In one embodiment of the invention the nanoparticles are metal nanoparticles which are selected from a group comprising gold, silver, titanium and platinum or the nanoparticles are magnetic metallic nanoparticles selected from Fe3O4.

However, metal nanoparticles, especially gold nanoparticles have been already described as signaling tools in many analytic/diagnostic applications. Different sizes and shapes of gold nanoparticles are for example used in lateral flow assays, the latter for generating duo-colored lateral flow tests¹⁸. Also peptide functionalized gold nanoparticles have been described in a variety of applications. In the detection of metal ions, several studies have appeared in the literature¹⁹⁻²². Whereby all described methods are based on colorimetric measurements of the spectral shift triggered by gold particle aggregation. The same physical measurement principle has been successfully applied to measure matrix metallo-proteinase matrilysin (MMP-7)²³, neurofenin 3 (ngn3)²⁴, bluetongue virus (BTV)-specific antibodies²⁵ or blood coagulation factor XIII²⁶. Even a cardiac Troponin-I assay, based on peptide functionalized gold nanorods, has been described²⁷. In contrast to the described assays the presented invention does not use the combination of optical and electronic properties of gold nanoparticles as signaling system. Instead this invention uses functionalized gold nanoparticles as charge carrying object, which are able to apply a field effect on semiconductor materials as signaling system. Accordingly, the invention uses the physical properties of the gold nanoparticles in a completely different and novel way.

In another embodiment of the invention the nanoparticles are semiconductor nanoparticles selected from a group comprising SiO₂.

In a further embodiment of the invention the nanoparticles are quantum dots selected from a group comprising CdSe/CdS, CdSe/ZnS, InAs/CdSe, ZnO/MgO, CdS/HgS, CdS/CdSe, ZnSe/CdSe, MgO/ZnO, ZnTe/CdSe, CdTe/CdSe and CdS/ZnSe.

In a most preferred embodiment of the invention the nanoparticles are selected from gold or Fe₃O₄ nanoparticles.

In a preferred embodiment of the invention the nanoparticles are charged to carry a positive or a negative charge. This is done by functionalizing the nanoparticles with SH-PEG-COOH or SH-PEG-NH₂. A nanoparticle functionalized with a SH-PEG-COOH group is charged to carry a negative charge, while a nanoparticle functionalized with a SH-PEG-NH₂ group is charged to carry a positive charge.

In a further preferred embodiment the nanoparticles are functionalized with SH-PEG-COOH groups to carry a negative charge or with SH-PEG-NH₂ to carry a positive charge.

The nanoparticles may be further modified with additional peptides which are characterized in that the peptide sequence represents polar and un-polar amino acids whereby the polar amino acids are homogeneously (only positively or negatively charged amino acids) in their charge. The peptides are further characterized in that they are between 4 and 25 amino acids long, whereby negatively charged peptides are exclusively coupled to SH-PEG-COOH functionalized nanoparticles, and whereas positively charged peptides are exclusively coupled to SH-PEG-NH₂ functionalized nanoparticles. Suitable peptides comprise a cysteine residue, for example CLDDD-OH or RRRLC-amid peptides are usable.

In one embodiment of the invention additional charged compounds are conjugated to the charged nanoparticles. Suitable charged compounds are selected from a group comprising charged peptides, nucleic acids like DNA and RNA, and are conjugated to the charged nanoparticles.

In a preferred embodiment of the invention the charged peptides additionally conjugated to the charged nanoparticles are between 4 and 25 amino acids long, preferably between 4 and 20 amino acids long.

In a preferred embodiment of the invention Cys-negative charged peptides or Cys-positive charged peptides are conjugated to the charged nanoparticles.

Therefore in one embodiment of the invention a CLDDD-OH is conjugated to a COOH functionalized nanoparticle.

In a further embodiment of the invention a RRRLC-amid is conjugated to a NH₂ functionalized nanoparticle.

Furthermore nucleic acids like DNA and RNA are suitable to be additionally conjugated to the charged nanoparticle, since their phosphate backbone is charged. Due to the charged phosphate backbone any sequence of DNA or RNA is suitable.

In one embodiment of the invention DNA or RNA is conjugated to the charged nanoparticles.

Modifying the charged nanoparticles with additional charged compounds as described above has the advantage that the field effect of the charged nanoparticle and therefore of the complex on the field effect transistor of the bio-sensing surface can be increased compared to the field effect of charged nanoparticles without modification. Therefore, the measurable difference between the field effect of the compound and the analyte to the field effect on the field effect transistor of the bio-sensing surface is increased as well.

Furthermore, a method of detecting an analyte by a biosensor is provided. Said method comprises a biosensor, wherein said biosensor comprises a bio-sensing surface which comprises a field effect transistor and a first binding molecule which is bonded to the surface of the field effect transistor. Furthermore the biosensor comprises a complex comprising second binding molecules which are conjugated to charged nanoparticles by linker molecules.

In a preferred embodiment the bio-sensing surface and the complex of the biosensor have the same features as described above for the biosensor according to the invention. In a further preferred embodiment the biosensor according to the invention is used in the method.

The method according to the invention comprises the following steps:
i. selecting a second binding molecule with a lower affinity to the first binding molecule compared to the analyte;
ii. conjugating the second binding molecules to charged nanoparticles;
iii. bonding the second binding molecules which are conjugated to charged nanoparticles to the bio-sensing surface;
iv. measuring the field effect of the charged nanoparticles to the field effect transistor by measuring the current in dependence of a voltage applied to the field effect transistor;
v. contacting the analyte with the bio-sensing surface and the charged nanoparticles which are conjugated to second binding molecules;
vi. measuring the change of the field effect acting on the field effect transistor by measuring the current in dependence of a voltage applied to the field effect transistor, wherein the second binding molecules conjugated to charged nanoparticles are partially or completely displaced by analytes due to the higher affinity of the analytes to the first binding molecules, thereby changing the field effect acting on the field effect transistor.

In step i) a second binding molecule is selected which is suitable for the measurement requirements. Therefore a second binding molecule is chosen with a lower affinity to the first binding molecule of the bio-sensing surface compared to the analyte. Which means the association constant Kₐ of the second binding molecule and the first binding molecule is less compared to the association constant of the analyte and the first binding molecule.

In step ii) the second binding molecules are conjugated to charged nanoparticles. In a preferred embodiment of the invention the second binding molecules and the charged nanoparticles are conjugated by a standard two step procedure.

Prior the conjugation of the second binding molecules with the charged nanoparticles, the nanoparticles are functionalized with carboxyl (COOH) or amino (NH₂) groups to carry a positive or a negative charge. The functionalization of metal nanoparticles is performed by the metal-thiol reaction using either SH-PEG-COOH or SH-PEG-NH2 heterobifunctional reagents with a molecular weight of 100-10,000 Da, preferable with 200-5,000 Da, more preferable with 300-3,000 Da, most preferable with 400-1,000 Da.

Heterobifunctional reagents can also be used to link nanoparticles to peptides or other molecules in a two- or three-step process that limits the degree of polymerization often obtained using homobifunctional crosslinkers. In a typical conjugation scheme, the nanoparticle is modified with a heterobifunctional compound using the crosslinker's most reactive or most labile end. The modified nanoparticle is then purified from excess reagent by centrifugation or by molecular weight cut-off columns. Most heterobifunctional linker contain at least one reactive group that displays extended stability in aqueous environments, therefore allowing purification of an activated intermediate before adding the second molecule (e.g peptide) to be conjugated. For instance, an NHS ester-maleimide heterobifunctional linker can be used to react with the amine groups of modified nanoparticles through its NHS ester end (the most labile functionality), while preserving the activity of its maleimide functionality. Since the maleimide group has greater stability in aqueous solution than the NHS ester group, a maleimide-activated intermediate may be created.

After a quick purification step, the maleimide end of the crosslinker can then be used to conjugate to a sulfhydryl containing molecule (e.g. a peptide via a cysteine residue).

Such multi-step protocols offer greater control over the resultant size of the conjugate and the molar ratio of components within the crosslinked product. The configuration or structure of the conjugate can be regulated by the degree of initial modification of the nanoparticle and by adjusting the amount of peptide added to the final conjugation reaction.

The third component of all heterobifunctional reagents is the cross-bridge or spacer that ties the two reactive ends together. Crosslinkers may be selected based not only on their reactivities, but also on the length and type of cross-bridge they possess. Some heterobifunctional families differ solely in the length of their spacer. The nature of the cross-bridge may also govern the overall hydrophilicity of the reagent.

For instance, polyethylene glycol (PEG)-based crossbridges create hydrophilic reagents that provide water solubility to the entire heterobifunctional compound. A few crosslinkers contain peculiar cross-bridge constituents that actually affect the reactivity of their functional groups. For instance, it is known that a maleimide group that has an aromatic ring immediately next to it is less stable to ring opening and loss of activity than a maleimide that has an aliphatic ring adjacent to it.

In a preferred embodiment of the invention heterobifunctional reagents are used to conjugate second binding molecules and charged metal particles.

Suitable heterobifunctional reagents are selected from a group comprising Mal-PEG-NH2, Mal-PEG-NHS, Mal-PEG-SulfoNHS and similar cross linkers with a molecular weight of 200-8,000 Da, preferable with 500-5,000 Da, more preferable with 1,000-4,000 Da, most preferable with 2,000-3,500 Da.

In a preferred embodiment of the invention Mal-PEG-NH₂ cross linker is used in combination with carboxyl functionalized nanoparticles.

In a further preferred embodiment of the invention Mal-PEG-NHS or Mal-PEG-SulfoNHS or similar cross linkers are used in combination with amino functionalized nanoparticles.

Particle size, surface composition, and density directly affect how a particle behaves in suspension. This in turn affects coupling protocols, especially in the handling and washing techniques used for particles during the conjugation process. Larger particles of micron size will generally settle over time just in normal gravity. As particle size decreases, however, a point is reached where a true colloidal suspension may occur, wherein the particles will not separate, no matter how long they sit in suspension. This typically happens when particle size gets to about 100 nm, and Brownian motion causes water molecules to collide with particles with high enough force-to-mass ratios to prevent them from settling under gravity. Many dense particles of less than 100 nm, such as silica, can still be separated from solution using a bench-top centrifuge; however, as particles approach the size of biological macromolecules, or around 10 nm, an ultracentrifuge would be required for separation.

For example Au nanoparticles down to a size of 15 nm can be purified using a standard laboratory centrifuge. Smaller Au nanoparticles down to 10 nm require ultracentrifugation. For even smaller Au nanoparticles down to 5 nm molecular weight cut-off columns for particle purification are necessary.

The charge repulsion effects between particles can be severely affected by the buffer and salt composition of the solution they are suspended in. Charges can be eliminated or neutralized by ionizable groups being protonated or unprotonated or by the concentration of ions in solution. For instance, lowering the pH of an aqueous solution below the pKa of the surface carboxylates will result in them being protonated. With most particles, especially ones having hydrophobic surfaces, this will cause particle aggregation due to loss of surface negative charge. Similarly, a high salt concentration can effectively mask the charge character of a carboxylated particle by having too many positively charged ions associated with the surface negative charges. Most particle types that are stable in suspension due to like charge repulsion can be made to aggregate if the pH is changed or the buffer or salt concentration is too high.

Part of the challenge of successfully working with small particles is to maintain optimal solution characteristics to keep the particles dispersed throughout the conjugation process. This includes all activation, coupling, and washing steps that are used to conjugate an affinity ligand, like the low-affinity molecule and subsequently use it in its intended application.

If individual particles in suspension are considered the equivalent of discrete molecules, then the molar concentration of a given particle suspension can be calculated based on the known particle diameter, density, and the mass of particles present. This allows particles to be treated similarly to other biomolecules with respect to determining concentration for conjugation purposes. However, there are important differences that should be recognized when working with particles as opposed to working with soluble macromolecules, like proteins. Since common commercial particles can vary in size from the molecular range (approximating the size of an antibody or −10-nm diameter) to a scale 1,000 times larger (or approaching the size of a cell at 10 μm), a change in diameter affects the concentration of particles as well as the effective concentration of surface functional groups present in suspension. Also potentially affected are the dispersion characteristics of particles as their size is changed (Suttiponparnit et al., 2011).

In general, as particle size decreases, the molar concentration of particles in a constant volume of solution increases (for a given mass of particles). For instance, a 1-mg quantity of 1-μm latex microspheres represents far fewer particles than a 1-mg amount of 50-nm nanoparticles. Thus, the effective molar concentration of nanoparticles in solution will be much greater than the concentration of the same mass of microparticles (if both are suspended at the same mass quantity and in same volume of solution). In addition, as the diameter decreases for a given mass of particles, the ratio of a particle's surface area to mass increases. This means that the total surface area available for conjugation on the nanoparticles is much greater than the total surface area present on the microparticles. If both particles contain the same functional groups on their surfaces for coupling affinity ligands (i.e., carboxylates), then for the same mass of particles the effective concentration of these functional groups in solution is much greater for the nanoparticles than the concentration of the same groups in a given solution for the microparticles (assuming both have about the same surface density or “parking area” of the carboxylate functional groups).

Thus, conjugation reactions performed with nanoparticles should take into account a potentially greater reactivity than the same reactions performed using microparticles, due to the higher effective concentration of functional groups present in solution for the nanoparticles. As particle size decreases and particle concentrations increase, the available surface area increases, and the effective concentration of reactive groups increases along with it.

Conjugation of the second binding molecules to the charged nanoparticles is therefore done in the following two-step procedure.

Step 1:

In order to conjugate peptides via a thiol-maleimide reaction on carboxylated or aminated nanoparticles, the nanoparticles have to be functionalized with a maleimide group. In case of SH-PEG-COOH functionalized nanoparticles, the functionalization is performed by a Mal-PEG-NH2 heterobifunctional reagents. In order to react the amino (NH2) group of the heterobifunctional cross linker with the carboxyl groups of the nanoparticle, an EDC/Sulfo NHS activation reaction is required. Therefore EDC and Sulfo-NHS are added to the SH-PEG-COOH functionalized nanoparticles for 15 min at room temperature. After activation of carboxyl groups the reaction mixture is purified from excess reagent by molecular weight cut-off columns and transferred into PBS (phosphate buffered saline) with pH 7.2. In particular, the activation of carboxylate particles using an EDC/Sulfo-NHS reaction will temporarily replace the negatively charged carboxylates with negatively charged sulfonates. The sulfonate groups on the Sulfo-NHS ester intermediates create a stronger negative charge on the particle surface than the original carboxylates. In some cases, the increase in negative charge repulsion can result in an inability to pellet the particles by centrifugation after the activation step even if the particles could be separated by centrifugation before activation. Therefore, in this invention molecular weight cut-off columns to purify reaction intermediates of EDC/sulfo-NHS reactions are used.

Immediately after purification a 10-100 fold molar excesses of Mal-PEG-NH2 heterobifunctional cross linker is added for 30 min at room temperature. The excess reagents are purified off by molecular weight cut-off columns.

In case nanoparticles functionalized with SH-PEG-NH₂ are used, the conjugation is performed by a Mal-PEG-NHS or Mal-PEG-Sulfo-NHS or similar heterobifunctional cross linker. The reaction takes place at a pH of 7.2-7.5 and will be performed for 15 min at room temperature. A pre-activation step is not required. A 10-100 fold molar excesses of Mal-PEG-NHS or Mal-PEG-Sulfo-NHS heterobifunctional cross linker is used. The excess reagents are purified off by molecular weight cut-off columns

Step 2:

The purified nanoparticle-PEG-Mal conjugate should be directly used for the peptide conjugation reaction. The reaction should take place at a pH between 6.5 and 7.5. Thiol-containing compounds, such as dithiothreitol (DTT) and beta-mercaptoethanol (BME), must be excluded from reaction buffers used with maleimides because they will compete for coupling sites. For example, if DTT were used to reduce disulfides, to make sulfhydryl groups available for conjugation, the DTT would have to be thoroughly removed using a desalting column before initiating the maleimide reaction. Interestingly, the disulfide-reducing agent TCEP does not contain thiols and does not have to be removed before reactions involving maleimide reagents. Excess maleimides can be quenched at the end of a reaction by adding free thiols. EDTA can be included in the coupling buffer to chelate stray divalent metals that otherwise promote oxidation of sulfhydryls (non-reactive). The conjugated peptides are used in a 10-1,000 fold molar excess. The reaction takes place at room temperature for 2-12 hours or at 4° C. for 4-24 hours. The excess reagents are purified off by molecular weight cut-off columns.

Furthermore, additional charged compounds may be added to the charged nanoparticles. This is done for compounds with a relative charge density greater than 10, for example for charged peptides, by a competitive reaction between the second binding molecule and the charged peptide with the maleimide functionalized charged Au nanoparticles. First the negative charged nanoparticles react with a NH₂-PEG-Mal linker (I). In a second reaction step a mixture of the second binding molecule and the charged peptide is added, in such a way that the charged peptide has a molar excess of 2-200 fold. In case of positively charged Au nanoparticles first the amino functionalized Au nanoparticles reacts with NHS-PEG-Mal heterobifunctional linker. In a second step the second binding molecule and the positive charged peptide is added, in such a way that the charged peptide has a molar excess of 2-200 fold.

According to step iii) of the method the second binding molecules which are conjugated to charged nanoparticles (complex) are added to the bio-sensing surface. The second binding molecule binds to the first binding molecule on the surface of the bio-sensing surface due to the affinity of both binding partners. The charged nanoparticles conjugated to the second binding molecules apply a field effect on the field effect transistor of the bio-sensing surface. Subsequently (step iv), the field effect is measured by measuring the current flow through the field effect transistor in dependence of a voltage applied to the field effect transistor.

In a next step the analyte is contacted with the bio-sensing surface. Due to the higher affinity of the analyte to the first binding molecule of the bio-sensing surface compared to the affinity of the second binding molecule to the first binding molecule of the bio-sensing surface, second binding molecules are partially or completely displaced by analytes due to the higher affinity of the analytes to the first binding molecules. The displacement of the second binding molecules by analytes is directly proportional to the concentration of the analyte. Due to the displacement of the second binding molecules and therefore also of the charged nanoparticles the field effect applied on the field effect transistor is now caused by the analytes.

In the last step of the method (step vi) according to the invention the change of the field effect acting on the field effect transistor is measured by measuring the current in dependence of a voltage applied to the field effect transistor. Therefore, the concentration of the analyte can be calculated by the change of the current in dependence of a voltage applied to the field effect transistor.

In one embodiment of the invention the concentration of the analyte is calculated by the change of the current in dependence of a voltage applied to the field effect transistor.

Advantageously, analytes can be detected which apply a low or even no measurable field effect on a field effect transistor due to the phenomenon of charge screening or Debye screening because the analyte is present in a solution with a high salt concentration. According to the present invention second binding molecules and consequently also charged nanoparticles are displaced by the analyte present in a solution, wherein the displacement is proportional to the concentration of the analyte in the solution. Due to the displacement the field effect applied on the field effect transistor decreases with increasing analyte concentration. Using this mechanism the concentration of the analyte in a solution can be measured.

However, also highly charged analytes can be detected by the present invention. Charged nanoparticles according to the invention have a relative charge density of approximately 30-150. The relative charge density of RNA is approximately 21 and of proteins even lower. Therefore, the field effect applied by the charged nanoparticles according to the invention is greater compared to the analyte in each case of interest. Accordingly, the filed effect applied by the analyte on the field effect transistor is lower compared to the field effect applied by the charged nanoparticles.

In a preferred embodiment of the invention the field effect of the analyte acting on a field effect transistor is lower compared to the field effect of the second binding molecule conjugated to a charged nanoparticle, preferably the field effect of the analyte acting on a field effect transistor is too low to be detectable.

Therefore, advantageously analytes present in solutions with high salt concentrations are detectable with the present invention. Especially analytes present in physiological solutions selected from blood, serum, saliva, stool, urine or plasma are detectable.

Accordingly, the invention describes a novel biosensor and a method comprising a bio-sensing surface and a complex which are able to detect biomarkers irrespectively of the physical/chemical properties. Further, universally all semiconductor materials in combination with different binding molecules can be used in the invention in combination with all relevant passivation/modification steps. Thereby, this invention solves a long standing problem for the entire biosensor and diagnostic field.

Additionally and in contrast to all published methods and procedures of the art, in this invention, the interaction between the nanoparticle and the first binding molecule, modulated by the conjugated second binding molecule, happens within a very well-defined affinity. This means that for any given analyte, a second binding molecule with a corresponding structure (e.g. peptide sequence) is selected in such a way that the affinity of the second binding molecule and the first binding molecule is lower compared to the affinity between the analyte and the first binding molecule. Thereby, a displacement reaction between the first binding molecule and the complex and therefore the charged nanoparticle, takes place as soon the analyte is added. Consequently, the applied field effect on the field effect transistor is altered in such a way that a significant measurement signal can be obtained.

In the following, the present invention is further described by 8 figures and 3 examples.

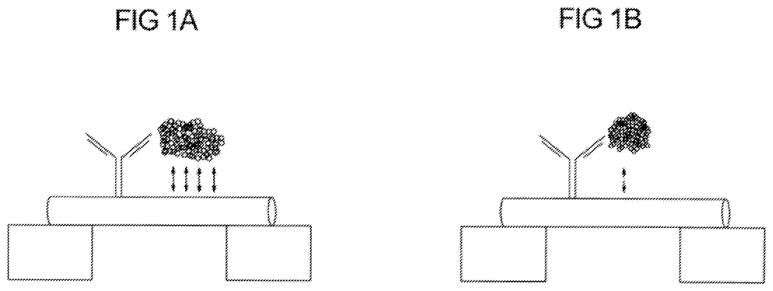

FIG. 1 illustrates FET biosensors which are state of the art;

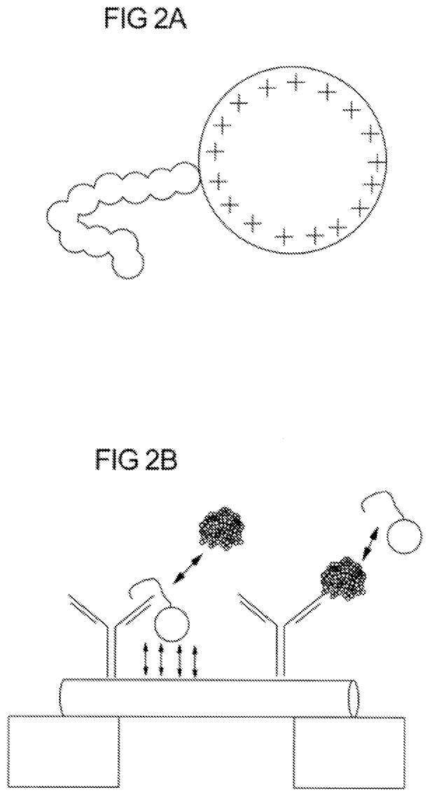

FIG. 2 (A) illustrates a charged nanoparticle and (B) illustrates the biosensor according to the invention;

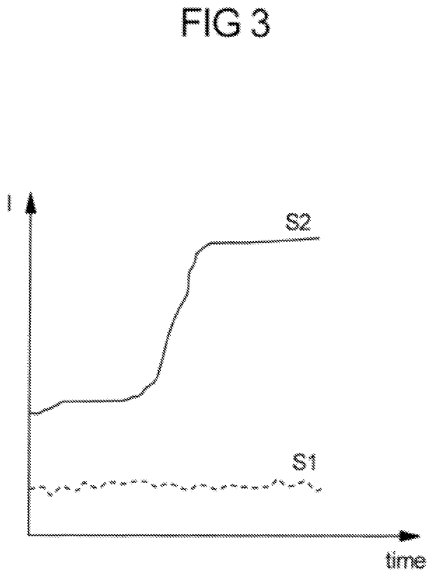

FIG. 3 illustrates the signals measurable with a field effect transistor of a complex according to the invention and an analyte present in a solution with a high salt concentration;

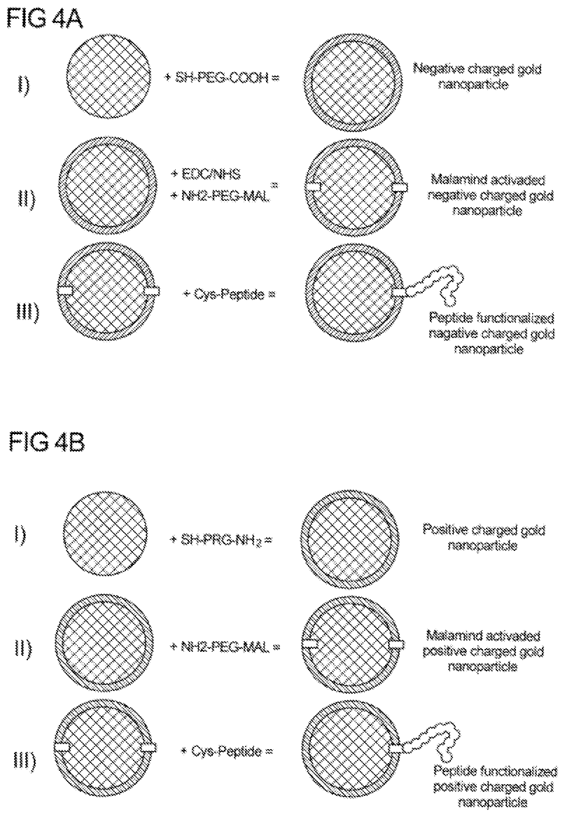

FIG. 4 (A) illustrates the functionalization of a gold nanoparticle to carry a negative charge and its conjugation with a second binding molecule, (B) illustrates the functionalization of a gold nanoparticle to carry a positive charge and its conjugation with a second binding molecule;

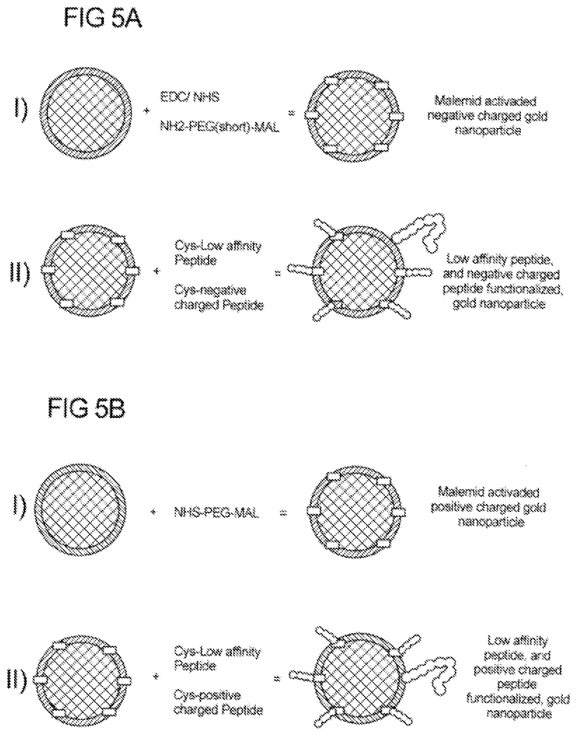

FIG. 5 (A) illustrates the functionalization of a negative charged gold nanoparticle conjugated to a second binding molecule which is functionalized with additional Cys-negative charged peptides, (B) illustrates the functionalization of a positive charged gold nanoparticle conjugated to a second binding molecule which is functionalized with additional Cys-positive charged peptides;

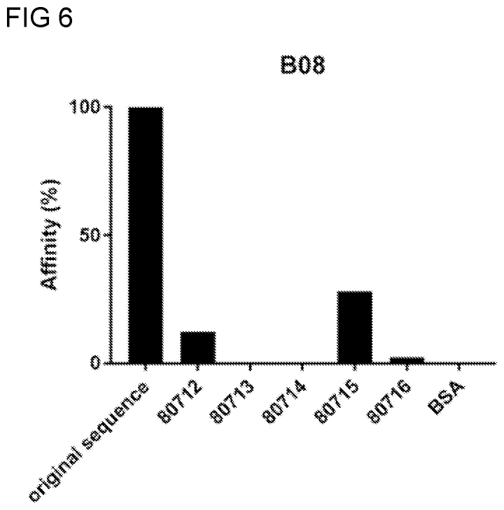

FIG. 6 shows the results of the affinity measurements of monoclonal mouse IgG1 anti human CRP antibody B08 against the biomarker CRP (SEQ ID NO: 1) and modified peptide sequences of SEQ ID NOs: 2 to 6;

FIGS. 1 (A) and (B) illustrate state of the art FET biosensors. FIG. 1 illustrates a semiconductor with an antibody acting as binding molecule on its surface. In FIG. 1 (A) an analyte is bound to the antibody and applies a measurable field effect on the field effect transistor. In several cases, especially when the analyte is present in solutions with high salt concentration, the analyte is bound to the antibody but no measurable field effect is applied to the semiconductor by the analyte, which is due to charge screening effects.

FIG. 2 (A) illustrates a charged nanoparticle which is conjugated to a second binding molecule. The second binding molecule is bound to a binding molecule on the surface of the field effect transistor, thereby the charged metal particle applies a field effect on the field effect transistor which is measurable (FIG. 2 (B)). If an analyte is added to the biosensor according to the invention the complex comprising the second binding molecule and the charged nanoparticle is displaced by the analyte on the binding site of the first binding molecule on the surface of the field effect transistor. Due to the displacement the field effect applied on the field effect transistor is altered. In case the analyte applies a low or even no field effect on the field effect transistor the measurable field effect on the field effect transistor is decreased. Since the displacement of the complex by the analyte is proportional to the concentration of the analyte, the change of the field effect is a measure for the concentration of the analyte in the solution. Therefore, especially analytes present in solutions with a high salt concentration or physiological solutions like blood, serum, saliva, stool, urine or plasma are detectable.

FIG. 3 illustrates the signals measurable with a field effect transistor of a complex according to the invention and an analyte present in a solution with a high salt concentration. The figure illustrates the current measured with a constant voltage of a field effect transistor in dependence of the time for two substances S1 and S2. S1 is a biomarker which is present in a high salt solution applying a week field effect on the field effect transistor (dashed line). Substance S2 is a complex according to the invention also present in a high salt solution. As can be seen a significantly higher current is measured with the field effect transistor for S2 (solid line).

In FIG. 4 (A) a gold nanoparticle is functionalized with a SH-PEG-COOH to carry a negative charge. Subsequently, the negative charged gold nanoparticle is conjugated to a Cys-peptide in a two-step procedure. Firstly, the carboxyl groups of the SH-PEG-COOH functionalized gold nanoparticle are activated by an EDC/NHS activation reaction. Afterwards the NH2-PEG-MAL heterobifunctional reagent is added and a maleimide activated negative charged nanoparticle is obtained. Secondly, a Cys-peptide is added and conjugated to the charged gold nanoparticle.

FIG. 4 (B) illustrates the procedure for a gold nanoparticle which is functionalized with a SH-PEG-NH₂ to carry a positive charge. Accordingly, the positive charged gold nanoparticle is conjugated to a Cys-peptide in a two-step procedure. Firstly, the NHS-PEG-MAL heterobifunctional reagents is added and a maleimide activated positive charged nanoparticle is obtained. Secondly, a Cys-peptide is added and conjugated to the charged gold nanoparticle.

Further charged compounds can be added to the complex of the invention. FIG. 5 (A) illustrates the two-step functionalization reaction of negative charged metal nanoparticles. First carboxylated metal nanoparticles are activated by EDC/SulfoNHS reaction and functionalized with an NH₂-PEG-MAL heterobifunctional cross linker. In a second reaction step a Cys-terminated peptide (a second binding molecule) is conjugated in parallel together with a negative charged Cys-peptide (like RRRLC-amid) to the maleimide group. Thereby additional negative charged groups are placed on the metal nanoparticle surface.

FIG. 5 (B) illustrates the two step functionalization reaction of positive charged metal nanoparticles. First aminated metal nanoparticles are functionalized with a SulfoNHS-PEG-MAL heterobifunctional cross linker. In a second reaction step a Cys-terminated peptide (a second binding molecule) is conjugated in parallel together with a positive charged Cys-peptide (like CLDDD-OH) to the maleimide group. Thereby additional positive charged groups are placed on the metal nanoparticle surface.

### EXAMPLES OF THE INVENTION

### Example 1—Functionalization of Gold Nanoparticles

The functionalization is performed by the gold (metal)-thiol reaction using either SH-PEG-COOH heterobifunctional reagents with a molecular weight of 400 Da. A 10 mg/ml SH-PEG-COOH (MW 634.77 g/mol) is added to 10 nM of gold nanoparticles having a diameter of 15 nm (functionalization works in the same way also for gold nanoparticles having a diameter of 20 nm, 10 nm or 5 nm) and incubated for 4-24 hours at RT. After the metal-thiol reaction is completed the Au nanoparticles are washed in water and PBS. The stability of the Au particles is determined by an UV/VIS spectral analysis. Stable Au nanoparticles show a high absorption at 520 nm and no absorption at 700 nm, whereas instable Au nanoparticles show a great absorption at 700 nm and a decreased absorption at 520 nm.

### Example 2—Two-Step Procedure to Conjugate a Second Binding Molecule and a Negative Charged Gold Nanoparticle

A gold nanoparticle is functionalized with SH-PEG-COOH to carry a negative charge. The charged gold nanoparticle shall be conjugated to a second binding molecule (which is a peptide) via a thiol-maleimide reaction in a two-step procedure according to the invention.

Step 1

The functionalization is performed by a Mal-PEG-NH2 heterobifunctional reagents. In order to react the amino (NH2) group of the heterobifunctional cross linker with the carboxyl groups of the nanoparticle, an EDC/Sulfo NHS activation reaction is required. Therefore 0.4 mg EDC and 1.1 mg Sulfo-NHS are added to 100 μl of 10 nM gold nanoparticles for 15 min at room temperature. After activation of carboxyl groups the reaction mixture is purified from excess reagent by molecular weight cut-off columns and transferred into PBS pH 7.2. Immediately after purification a 10-100 fold molar excesses of Mal-PEG-NH2 heterobifunctional cross linker is added for 30 min at room temperature. The excess reagents are purified off by molecular weight cut-off columns.

Step 2

The purified nanoparticle-PEG-Mal conjugate is directly used for the peptide conjugation reaction. The reaction takes place at a pH between 6.5 and 7.5. Thiol-containing compounds, such as dithiothreitol (DTT) and beta-mercaptoethanol (BME), are excluded from reaction buffers used with maleimides because they will compete for coupling sites.

DTT, which is used to reduce disulfides, to make sulfhydryl groups available for conjugation is thoroughly removed using a desalting column before initiating the maleimide reaction. Since the disulfide-reducing agent TCEP does not contain thiols it is not removed before reactions involving maleimide reagents. Excess maleimides are quenched at the end of a reaction by adding free thiols. EDTA is included in the coupling buffer to chelate stray divalent metals that otherwise promote oxidation of sulfhydryls (non-reactive). The conjugated peptides are added in a 10-1,000 fold molar excess. The conjugation reaction takes place at room temperature for 2-4 hours. The excess reagents are purified off by molecular weight cut-off columns.

### Example 3—Two-Step Procedure to Conjugate a Second Binding Molecule in Parallel Together with Additional Negative Charged Molecules to a Negative Charged Gold Nanoparticle

A gold nanoparticle is functionalized with SH-PEG-COOH to carry a negative charge. The charged gold nanoparticle shall be conjugated to a second binding molecule (which is a peptide) and to an additional negative charged molecule (which is a peptide of the sequence: RRRLC-OH) via a thiol-maleimide reaction in a two-step procedure according to the invention.

Step 1

The functionalization is performed by a Mal-PEG-NH2 heterobifunctional reagents. In order to react the amino (NH2) group of the heterobifunctional cross linker with the carboxyl groups of the nanoparticle, an EDC/Sulfo NHS activation reaction is required. Therefore 0.4 mg EDC and 1.1 mg Sulfo-NHS are added to 100 μl of 10 nM gold nanoparticles for 15 min at room temperature. After activation of carboxyl groups the reaction mixture is purified from excess reagent by molecular weight cut-off columns and transferred into PBS pH 7.2. Immediately after purification a 10-1,000 fold molar excesses of Mal-PEG-NH2 heterobifunctional cross linker is added for 30 min at room temperature. The excess reagents are purified off by molecular weight cut-off columns.

Step 2

The purified nanoparticle-PEG-Mal conjugate is directly used for the peptide conjugation reaction. The reaction takes place at a pH between 6.5 and 7.5. Thiol-containing compounds, such as dithiothreitol (DTT) and beta-mercaptoethanol (BME), are excluded from reaction buffers used with maleimides because they will compete for coupling sites.

The conjugated peptides are added in a 10-1,000 fold molar excess whereby the additional negative charged molecule has a 5-20 fold molar excess compared to the second binding molecule. This means if the second binding molecule is used in 10 fold molar excess, the additional negative charged molecule has a 50-200 fold molar excess compared to the gold nanoparticle concentration. The conjugation reaction takes place at room temperature for 2-4 hours. The excess reagents are purified off by molecular weight cut-off columns.

### Example 4—Coupling of a First Binding Molecule on swCNTs

The single walled CNT (swCNT) network is present on a biosensor surface and shall be functionalized with a first binding molecule (an antibody).

First a 1 mM 1-pyrenebutric acid solution in EtOH is incubated for 1-24 hours at room temperature. The excess reagent is purified off by washing the sensor 3 times with EtOH followed by a subsequent 3 times washing step with water. The functionalization of the antibody is performed by coupling the antibody amino groups with the carboxyl group of the 1-pyrenebutric acid. Consequently the carboxyl groups of the 1-pyrenebutric acid have to be activated by an EDC/Sulfo NHS activation reaction. Therefore 0.4 mg EDC and 1.1 mg Sulfo-NHS are added to 1 ml of an amino free buffer like PBS (pH 6.0). The activation reaction takes place for 15 minutes at room temperature.

Directly after the activation reaction the antibody is added in a concentration of 1-0.1 mg/ml to the biosensor at pH 7.2-8.0 for 1-4 hours at room temperature. The excess antibody is purified off by washing the sensor 3 times with PBS pH 7.2.

### Example 5—Test of Sensor Functionality

The current sensor chips are conducted by crocodile clamps to a dual-channel source meter (Keithley 2612B). The samples are applied by a pipet. Sample volumes are varied between 10 and 50 μl.

As gate electrode a Ag/AgCl electrode operating in a top gate setting was used. A feedback circuit was also implemented, which measures constantly the applied gate current and regulates the gate current voltage if necessary.

In a pre-test the sensitivity to fluids with different pH-values was tested. As result it was found that the sensor reacts very strongly and reliably to a change between pH 6 and pH 7 solutions.

To test the measurement set up and the general sensor functionality different pH PBS buffers were subsequently applied on the sensor surface. Therefore, a PBS solution (pH 7) was mixed with 20 nm Au nanoparticles (Au-NP) and a concentration of 2.4 pM. The same PBS solution without Au-NP served as reference. It could be shown that there is a significant sensor response when the two fluids are exchanged cyclically. This confirms that the semiconductor sensor is influenced by low concentrations of Au-NP.

### Example 6—Measurement of the Biomarker C-Reactive Protein (CRP)

A complex comprising a second binding molecule that is coupled to Au nanoparticle via a linker has been produced as described in example 1. 5 nM Au particles with 10 functionally coupled peptides per Au particle were used in the experiment.

The biosensor was functionalized as described in example 4, in this experiment with the monoclonal mouse IgG1 anti human CRP antibody B08. The affinity of the antibody was first tested against CRP (SEQ ID NO: 1) and the modified peptide sequences of SEQ ID NOs: 2 to 6. Sequences are shown in the following table:

| | Amino acid | |
|---|---|---|
| Peptide-ID | sequence | SEQ ID NO: |
| Original sequence of | CVFPKESD | 1 |
| CRP | | |
| 80712 | CAFPKESD | 2 |
| 80713 | CVFPRESD | 3 |
| 80714 | CVFPKDSD | 4 |
| 80715 | CVFPKETD | 5 |
| 80716 | CVYPKESD | 6 |

The results of the affinity measurements are shown in FIG. 6. The subsequent displacement measurements were performed with the peptide 80715 (SEQ ID NO: 5).

The Au nanoparticles are bound to the CRP-specific antibody via a peptide and exert a field effect on the semiconductor. If the biomarker (in this case CRP) is present in the blood sample, it can displace the nanoparticle and thus annul the field effect.

Results of the measurement of a displacement reaction: The current/voltage curve shows the change in the transistor property (by annulling the field effect). First PBS without biomarker was added, then the concentration of the biomarker CRP in PBS was gradually increased. By adding different concentrations of the biomarker CRP, the current-voltage curve of the transistor has changed accordingly. The following CRP concentration s were used: 381 fM, 3 pM, 24 pM, 195 pM, 1.56 nM, 12.5 nM, 100 nM and 800 nM. The voltage changes measured are shown in the following table:

| | |
|---|---|
| CRP concentration | ΔV (V) |

| | | |
|---|---|---|
| 381 | fM | −0.009 |
| 3 | pM | −0.013 |
| 24 | pM | −0.016 |
| 195 | pM | −0.017 |
| 1.56 | nM | −0.020 |
| 12.5 | nM | −0.022 |
| 100 | nM | −0.024 |
| 800 | nM | −0.027 |

At constant current, changes in the biomarker concentration were measured as voltage changes. It was found that there is a nearly linear relationship between voltage change (ΔV in V) and CRP concentration. The biomarker CRP could be reliably detected in a concentration range between 800 nM to 381 fM. Reaction times were 10 minutes, measurements were performed in PBS (i.e. 150 mM salt concentration).

### LITERATURE

1. Schenk, J. U.S. Pat. No. 4,238,757A. (1976).
2. G. G. Liversidge, K. C. Cundy, J. F. Bishop, D. A. C. U.S. Pat. No. 5,192,507A. (1987).
3. Andoy, N., Filipiak, M., Gutiérrez-Sanz, Ó., Vetter, D. & Tarasov, A. Graphene-Based Electronic Immunosensor with Femtomolar Detection Limit in Whole Serum. Adv. Mater. Technol. 1-12 (2018). doi:10.1002/admt.201800186
4. Evans, T. C., Gavrilovich, E., Mihai, R. C. and Isbasescu, I., E. L. US2008/0283875 A1. (2005).
5. Evans, T. C., Gavrilovich, E., Mihai, R. C. and Isbasescu, I., E. L. (12) Patent Application Publication (10) Pub. No.: US 2006/0222585 A1 FIG. 1. Gridbridge 002, 354 (2015).
6. Filipiak, M. S. et al. Highly sensitive, selective and label-free protein detection in physiological solutions using carbon nanotube transistors with nanobody receptors. Sensors Actuators, B Chem. 255, 1507-1516 (2018).
7. Gutierrez-Sanz, Ó., Andoy, N. M., Filipiak, M. S., Haustein, N. & Tarasov, A. Direct, Label-Free, and Rapid Transistor-Based Immunodetection in Whole Serum. ACS Sensors 2, 1278-1286 (2017).
8. Jae-Ho Kim, Sung-Wook Choi, Jae-Hyeok Lee, G. & Nam, H. US 2012/0073992. (2010).
9. Sadlowski, C. M. et al. Graphene-based biosensor for on-chip detection of Bio-orthogonally Labeled Proteins to Identify the Circulating Biomarkers of Aging during Heterochronic Parabiosis. Lab Chip (2018). doi:10.1039/C8LC00446C
10. Sanchez-Tirado, E., Gonzalez-Cortes, A., Yáñez-Sedeño, P. & Pingarrón, J. M. Carbon nanotubes functionalized by click chemistry as scaffolds for the preparation of electrochemical immunosensors. Application to the determination of TGF-beta 1 cytokine. Analyst 141, 5730-5737 (2016).
11. Star, A., Gabriel, J. C. P., Bradley, K. & Grüner, G. EP1645871B1. (2003).
12. Tilmaciu, C.-M. & Morris, M. C. Carbon nanotube biosensors. Front. Chem. 3, 1-21 (2015).
13. Kim, J. P., Lee, B. Y., Hong, S. & Sim, S. J. Ultrasensitive carbon nanotube-based biosensors using antibody-binding fragments. Anal. Biochem. 381, 193-198 (2008).
14. Wenchuang Hu, R. & Tian, Suresh Regonda, K. B. T. US2013/0291627A1. (2010).
15. Yavuz, A. A., Glas, B., Ihle, M., Hacioglu, H. & Wehefritz, K. US 2006/0205013 A1. (2005).
16. Friederike J. Gruhl, B. E. R. and K. L. Biosensors for Diagnostic Applications Friederike. Adv Biochem Engin/Biotechnol (2011). doi:10.1007/10_2011_130
17. Zhou, Y., Fang, Y. & Ramasamy, R. Non-Covalent Functionalization of Carbon Nanotubes for Electrochemical Biosensor Development. Sensors 19, 392 (2019).
18. Petrakova, A. V., Urusov, A. E., Zherdev, A. V. & Dzantiev, B. B. Gold nanoparticles of different shape for bicolor lateral flow test. Anal. Biochem. 568, 7-13 (2019).
19. Si, S., Kotal, A. & Mandal, T. K. One-dimensional assembly of peptide-functionalized gold nanoparticles: An approach toward mercury ion sensing. J. Phys. Chem. C 111, 1248-1255 (2007).
20. Slocik, J. M., Zabinski, J. S., Phillips, D. M. & Naik, R. R. Colorimetric response of peptide-functionalized gold nanoparticles to metal ions. Small 4, 548-551 (2008).
21. Wang, C., Wang, J., Liu, D. & Wang, Z. Gold nanoparticle-based colorimetric sensor for studying the interactions of β-amyloid peptide with metallic ions. Talanta 80, 1626-1631 (2010).
22. Jongjinakool, S., Palasak, K., Bousod, N. & Teepoo, S. Gold nanoparticles-based colorimetric sensor for cysteine detection. Energy Procedia 56, 10-18 (2014).
23. Chen, P., Selegärd, R., Aili, D. & Liedberg, B. Peptide functionalized gold nanoparticles for colorimetric detection of matrilysin (MMP-7) activity. Nanoscale 5, 8973-8976 (2013).
24. Yuan, Y., Zhang, J., Zhang, H. & Yang, X. Label-free colorimetric immunoassay for the simple and sensitive detection of neurogenin3 using gold nanoparticles. Biosens. Bioelectron. 26, 4245-4248 (2011).
25. Saxena, V. K. et al. Functionalizing gold nanoparticles with bluetongue virus multiple peptide antigens utilizing gold-thiol interaction: A novel approach to develop pen side test. Res. Vet. Sci. 93, 1531-1536 (2012).
26. Chandrawati, R. & Stevens, M. M. Controlled assembly of peptide-functionalized gold nanoparticles for label-free detection of blood coagulation Factor XIII activity. Chem. Commun. 50, 5431-5434 (2014).
27. Tadepalli, S. et al. Peptide Functionalized Gold Nanorods for the Sensitive Detection of a Cardiac Biomarker Using Plasmonic Paper Devices. Sci. Rep. 5, 1-11 (2015).
28. Xiaochen Dong et al: Electrical Detection of Femtomolar DNA via Gold-Nanoparticle Enhancement in Carbon-NanotubeNetwork Field-Effect Transistors, ADVANCED MATERIALS, vol. 20, no. 12, 18 Jun. 2008 (2008 Jun. 18), pages 2389-2393.
29. Kim J P et al: Enhancement of sensitivity and specificity by surface modification of carbon nanotubes in diagnosis of prostate cancer based on carbon nanotube field effect transistors, BIOSENSORS AND BIOELECTRONICS, ELSEVIER SCIENCE LTD. UK, AMSTERDAM, NL, vol. 24, no. 11, 15 Jul. 2009 (2009 Jul. 15), pages 3372-3378.
30. Shun Mao et al: Highly sensitive protein sensor based on thermally-reduced graphene oxide field-effect transistor. NANO RESEARCH, vol. 4, no. 10, 21 May 2011 (2011 May 21), pages 921-930.
31. Hsiao-Chien Chen et al: Magnetic-Composite-Modified Polycrystalline Silicon Nanowire Field-Effect Transistor for Vascular Endothelial Growth Factor Detection and Cancer Diagnosis, ANALYTICAL CHEMISTRY, vol. 86, no. 19, 15 Sep. 2014 (2014 Sep. 15), pages 9443-9450.
32. Ana de Moraes et al: Recent Trends in Field-Effect Transistors-Based lmmunosensors. CHEMOSENSORS, vol. 4, no. 4, 21 Dec. 2016 (2016 Dec. 21), page 20.
33. Song Liu et al: Carbon nanomaterials field-effect-transistorbased biosensors. NPG ASIA MATERIALS, vol. 4, no. 8, 1 Aug. 2012 (2012 Aug. 1), pages e23-e23.

1. A biosensor for detecting analytes comprising a bio-sensing surface which comprises a field effect transistor and a first binding molecule which is bonded to the surface of the field effect transistor;
and a complex comprising second binding molecules which bind to the first binding molecule and which are conjugated to charged nanoparticles by linker molecules, wherein,
at least one second binding molecule is conjugated to one charged nanoparticle;
the at least one second binding molecule conjugated to a charged nanoparticle interacts with the first binding molecule wherein the charged nanoparticle is configured to apply a field effect on the field effect transistor;
the affinity of the at least one second binding molecule to the first binding molecule is adaptable such that the first binding molecule releases the complex comprising the at least one second binding molecule in presence of the analyte;
and the field effect transistor is configured such that the current measured in dependence of a voltage applied to said field effect transistor is changed due to displacement of the complex comprising the at least one second binding molecule from the first binding molecule by the analyte.

2. A biosensor according to claim 1, wherein the complex comprises
a charged nanoparticle selected from a group consisting of metallic nanoparticles, semiconductor nanoparticles, quantum dots or non-metallic nanoparticles, wherein the nanoparticles are charged to carry a positive or negative charge;
at least one linker molecule selected from a group consisting of a bond, alkyl, polyethylene glycol (PEG), polyamide, peptide, carbohydrate, oligonucleotide or polynucleotide; and
at least one second binding molecule selected from a group consisting of proteins, peptides, nucleic acids or synthetic components.

3. A biosensor according to claim 1, wherein one second binding molecule is conjugated to one charged nanoparticle.

4. A biosensor according to claim 1, wherein the affinity of the at least one second binding molecule to the first binding molecule is less compared to the affinity of the analyte to the first binding molecule.

5. A biosensor according to claim 1, wherein the first binding molecule is selected from proteins, peptides, nucleic acids or antibodies and fragments thereof.

6. A biosensor according to claim 1, wherein the nanoparticle is a metallic nanoparticle and is selected from a group consisting of gold, silver, titanium and platinum, or the nanoparticles are magnetic metallic nanoparticles selected from Fe₃O₄, or wherein the nanoparticle is a semiconductor nanoparticle selected from a group consisting of SiO₂ or the nanoparticle is a quantum dot selected from a group consisting of CdSe/CdS, CdSe/ZnS, InAs/CdSe, ZnO/MgO, CdS/HgS, CdS/CdSe, ZnSe/CdSe, MgO/ZnO, ZnTe/CdSe, CdTe/CdSe and CdS/ZnSe.

7. A biosensor according to claim 6, wherein the nanoparticle is functionalized with SH-PEG-COOH to carry a negative charge; or
wherein the nanoparticle is functionalized with SH-PEG-NFh to carry a positive charge.

8. A biosensor according to claim 1, wherein additional charged compounds are conjugated to the charged nanoparticle.

9. A biosensor according to claim 8, wherein charged compounds selected from charged peptides or nucleic acids are conjugated to the charged nanoparticle.

10. A biosensor according to claim 1, wherein Cys-negative charged peptides or Cys-positive charged peptides are conjugated to the charged nanoparticle.

11. A method of detecting an analyte with a biosensor wherein the method comprises the steps of
i. providing a biosensor with a bio-sensing surface which comprises a field effect transistor and a first binding molecule which is bonded to the surface of the field effect transistor;
ii. selecting a second binding molecule with a lower affinity to the first binding molecule compared to the analyte;
iii. conjugating the second binding molecules to charged nanoparticles via linker molecules;
iv. bonding the second binding molecules, which are conjugated to charged nanoparticles via linker molecules, to the first binding molecule of the biosensing surface;
v. measuring the field effect of the charged nanoparticles to the field effect transistor by measuring the current in dependence of a voltage applied to the field effect transistor;
vi. contacting the analyte with the bio-sensing surface and the charged nanoparticles which are conjugated to second binding molecules;
vii. measuring the change of the field effect acting on the field effect transistor in presence of the analyte by measuring the current in dependence of a voltage applied to the field effect transistor,
wherein the second binding molecules conjugated to charged nanoparticles are partially or completely displaced by analytes due to the higher affinity of the analytes to the first binding molecules, thereby changing the field effect acting on the field effect transistor.

12. The method of detecting an analyte by a biosensor according to claim 11, wherein the concentration of the analyte is calculated by the change of the current in dependence of a voltage applied to the field effect transistor.

13. The method according to claim 11, wherein the second binding molecules and the charged nanoparticles are conjugated by a standard two step procedure.

14. The method according to claim 11, wherein the analyte is present in a physiological solution selected from blood, serum, saliva, urine, stool or plasma.

15. The method according to claim 11, wherein the field effect of the analyte acting on a field effect transistor is lower compared to the field effect of the second binding molecule conjugated to a charged nanoparticle, wherein the field effect of the analyte and of the charged nanoparticle on the field effect transistor is determined by measuring the current in dependence of a voltage applied to the field effect transistor.

16. The biosensor according to claim 1, wherein said biosensor is configured to detect an analyte which is present in a physiological solution selected from blood, serum, saliva, urine stool or plasma.

17. The biosensor according to claim 1, wherein the field effect of the analyte acting on a field effect transistor is lower compared to the field effect of the second binding molecule conjugated to a charged nanoparticle, wherein the field effect of the analyte and of the charged nanoparticle on the field effect transistor is determined by measuring the current in dependence of a voltage applied to the field effect transistor.
